(19) United States
(12) Patent Application Publication
(10) Pub. No.: US 2006/0029690 A1
(43) Pub. Date: Feb. 9, 2006

(54) Buckwheat compound and method of obtaining the same for treatment of hyperglycemia

(76) Inventors: Chi Wai King Kwong, Hong Kong (HK); Guixing Ren, Hong Kong (HK); Chi Wan Vanessa Lau, Hong Kong (HK); Chong Tai Lam, Hong Kong (HK); Zhu Fang Shen, Beijing (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/913,854

(22) Filed: Aug. 6, 2004

(51) Int. Cl. A61K 36/185

(52) U.S. Cl. 424/776

(57) ABSTRACT

A process for obtaining a compound from buckwheat and its use as a pharmaceutical treatment are disclosed. Methods of treatment using extracts of buckwheat are also disclosed.

### FIELD OF INVENTION

The present invention is related to the extract of tartary buckwheat bran, method of extraction and the use of the extract as anti-diabetic agent.

### DESCRIPTION OF THE RELATED ART

Common and tartary buckwheat have been grown in many countries around the world as a source of food. The flour and bran of both types of buckwheat are proven to contain useful nutrients, such as protein, lipid, starch, dietary fibre and vitamins B1, B2 and B6.

Buckwheat products are known for their resistant starch (Skrabanja, Laerke, & Kreft, 1998; Skrabanja, Liljeberg, Kreft, & Bjorck, 2001) and as an important source of antioxidative substances (Kreft, Bonafaccia, & Zigo, 1994; Kreft, Skrabanja, Ikeda, Ikeda, & Bonafaccia, 1996), trace element, and dietary fibre (Steadman, Burgoon, Lewis, Edwardson, & Obendorf 2001). Buckwheat protein products have been associated preventive nutrition. They are associated with retardation of mammary carcinogenesis by lowering serum estradiol, and with suppression of colon carcinogenesis by reducing cell proliferation (Kayashita, Shimaoka, Nakajoh, Kishida, & Kato, 1999; Liu et al., 2001). There are, however, only a few reports on the technological quality of buckwheat. Buckwheat is also known as effective in modulating blood sugar and blood lipids levels. However, the efficiency thereof is not very satisfactory.

It is therefore an object of the present invention to provide compound and improved method of production of compounds for the treatment of hyperglycemia.

### SUMMARY OF INVENTION

In accordance with the object of the present invention, there is provided in one aspect an active component in buckwheat for the treatment of hyperglycemia. In the preferred embodiment, the active component is obtained by extraction of buckwheat bran using about 5-90% (w/v) ethanol. In another preferred embodiment, the active component is obtained particularly in tartary buckwheat. In yet another preferred embodiment, the ethanol concentration is about 10-30% (w/v). In the most preferred embodiment, the ethanol concentration is about 20% (w/v).

According to another aspect of the invention, there is provided a novel anti-diabetic formulation comprising an effective amount of the extract obtained from tartary buckwheat bran, optionally together with additives, pharmaceutically accepted carriers, diluents or excipients. The concentration of the extract in the formulation may be 1 to 100% by wt.

Another aspect of the present invention is a method of extracting a biologically active component from buckwheat comprising extraction of buckwheat bran using about 10-90% (w/v) ethanol. In the preferred embodiment, the active component of the present method is obtained particularly from tartary buckwheat. In another preferred embodiment, the ethanol concentration is about 10-30% (w/v). In the most preferred embodiment, the ethanol concentration is about 20% (w/v).

According to a further aspect of the present invention, an extract from buckwheat bran obtained by the method described above is used for the preparation of a medicament or nutritional supplement for reducing blood glucose concentration.

According to yet a further aspect of the present invention, there is provided a method of reducing blood glucose concentration in humans by administering an effective dose of an extract from buckwheat bran obtained by the method described above.

An additional aspect of the invention comprises a method for obtaining an anti-hyperglycemia agent, comprising the steps of:
(a) drying fresh seeds of tartary buckwheat at ambient temperature, preferably around 10 degree Celsius;
(b) separating the bran from dried seeds by grinding;
(c) extracting the bran with ten fold of about 10-30% ethanol (w/v) for a period ranging from about 24-120 hrs. at a temperature in a range of about 10-30 degree C., shaking or stirring occasionally during extraction and transferring the liquid extract to a centrifuge bottle; and
(d) centrifuging at a speed in a range of about 1000-3000 rpm/min for about 5-30 min to obtain supernatant liquid, then filtering through about 100-300 mesh sieve filter, followed by evaporating the filtered liquid to obtain a residue at reduced pressure with temperature of about 60-80 degree C., and drying the residue with vacuum-freezing drier to obtain dried residue.

Additional aspects of the invention include methods of treating hyperglycemia in an individual comprising the administration of a formulation comprising extract from tartary buckwheat bran to said individual in a dosage of about 0.1 to 5 g/kg body weight per day. In a preferred embodiment, a method of treatment for an adult mammal comprises the administration of a formulation comprising extract from tartary buckwheat bran to said mammal at a dosage of about 5 to 250 mg/kg body weight per day. In an additional preferred embodiment, the dosage of a formulation comprising extract from tartary buckwheat bran used to treat an individual varies according to the severity of the condition being treated and the pharmacological activity of the formulation being used.

In some embodiments of the present invention, a product obtained by extracting buckwheat with about 10-30% ethanol is provided. The amount of ethanol can be, for example, at about 20%. The buckwheat can be, for example, tartary buckwheat or buckwheat bran. The extraction may occur by concentration of the product by evaporation or by drying. In some embodiments, the product can be obtained by centrifugation after the ethanol has been placed in contact with the buckwheat. The product can also be used for the preparation of a medicament for lowering blood glucose.

In additional embodiments of the present invention, a pharmaceutical composition for lowering blood glucose having a extract of buckwheat bran obtained by extraction with about 10% to 30% ethanol is provided.

In further embodiments of the present invention, a nutritional supplement for lowering blood glucose having an extract of buckwheat bran obtained by extraction with about 10% to 30% ethanol is provided.

In additional embodiments of the present invention, a method for treating diabetic symptoms in an individual by the administration of an effective dose of an extract of buckwheat to the individual is provided, where the extract is obtained by extraction of buckwheat with about 10% to 30% ethanol. The ethanol can be at a level of about 20%. The diabetic symptoms may be, for example, hyperglycemia, glucose intolerance, or hyperlipidemia. The hyperlipidemia can be hypercholesterolemia or hypertriglyceridemia. The treatment may manifest, for example, as lower fasting blood glucose, lower non-fasting blood glucose, higher levels of superoxide dismutase activity, higher catalase activity levels, lowering peak glucose levels and increasing the rate of falling blood glucose levels.

In yet additional embodiments of the present invention, a method for isolating an anti-hyperglycemic agent from buckwheat is provided, by drying seed from the buckwheat, isolating the bran from the seed, extracting the agent from the bran with about 10% to 30% ethanol, and processing the ethanol to isolate the anti-hyperglycemic agent. The buckwheat can be, for example, tartary buckwheat. The drying of the seed can take place, for example, at a temperature of about 10 degrees Celsius. The alcohol can be, for example, about 10% to 30% ethanol (w/v). The ethanol can have a concentration of about 20 % (w/v). The processing can involve centrifuging the ethanol to obtain a supematant liquid, evaporating the supematant liquid to obtain a residue, and drying the residue. The centrifugation can be performed at speeds of, for example, about 1000 to about 3000 RPM for about 5 to about 30 minutes. The method may also include a filtering step after centrifugation. The filtering step can be performed, for example, with an about 100 to about 300 mesh sieve filter.

### DETAILED DESCRIPTION OF THE PREFERRED EMBODIMENT

The experiments to identify a biologically active component for reducing blood sugar levels were performed initially using various groups of compounds. These groups included flavonoids and other solvent-extracted fractions obtained from buckwheat.

The experiments were performed in two stages. In the first stage, the effectiveness of (1) a 20% ethanol extract of tartary buckwheat; (2)a 60% ethanol extract of tartary buckwheat; and (3) flavonoid(s) purified from tartary buckwheat were tested on alloxan diabetic mice and STZ (streptozocin) diabetic rats. The negative and positive controls used for comparison were water (negative control group) and metformin (positive control group) respectively. In the second stage, the effectiveness of 20% ethanol extract at different doses was further tested on STZ diabetic rats, in comparison with water (negative control group) and metformin (positive control group).

As stated earlier, the extract is obtained from tartary buckwheat bran. The said extract is used to prepare the anti-diabetic formulation. The formulation may be prepared according to any method known in the art. The formulation may be intended for oral, parenternal or other uses. The formulation for oral use may be in the form of granules, particles, powders, tablets, capsules, liquid syrup, etc. In order to prepare such formulation, any pharmaceutically acceptable organic or inorganic, solid or liquid carrier, excipient, diluent may be used. The formulation may also contain sweetening agent, flavoring agent, colouring and preserving agents in order to provide pharmaceutically elegant and palatable preparations. Tablets containing the active ingredients, are prepared using the extract from tartary buckwheat bran in combination with non-toxic pharmaceutically acceptable carriers or additives.

Another aspect of the invention features methods for evaluating the ability of buckwheat extracts to treat disease conditions. After extraction of material from buckwheat, components of the material are separated from one another by any one or more of a variety of techniques, essentially creating two or more fractions of extract material. Methods of separating the protein, mineral and carbohydrate materials that can be present in such extracts are known to those with skill in the art. Examples of such methods include precipitation, centrifugation, filtration, PAGE, SDS-PAGE, high performance liquid chromatography, size exclusion chromatography, ion exchange chromatography, affinity chromatography and immunoassay. In these method examples, component molecules of extracts are separated from one another based on any one or more of numerous physical and chemical properties, including, but not limited to, molecular size, charge, affinity for other molecules, including hydrophobicity, solublity, molecular shape and molecular structure. Once separated from the initial extract, fractions of extracts can be tested for their ability to affect biochemical properties of clinical samples and disease symptoms. The examples below provide some exemplary methods for testing the properties of extracts and fractions of extracts. It would be evident to one with skill in the art that any number of different tests and assays could be used to evaluate the properties of extracts and fractions of extracts.

In the following animal study experiments, performed at the Institute of Materia Medica of The Chinese Academy of Medical Science, the glucose as well as the reagents were administered orally into the mice and rats.

### EXAMPLE 1

### Determination of Effectiveness of Tartary Buckwheat Extracts

1.1 Preparing Tartary Buckwheat Extracts

1.1.1 20% Ethanol Extracts

The bran was provided by Shanxi GAP cultivation site. Food Grade Ethanol was purchased from CSA Distilleries. Deionized water was provided from in house water system.

Buckwheat concentrates were produced by soaking the bran of tartary buckwheat in ten fold volume of 20% ethanol for 72 hours. The extract was stirred occasionally within the set period (one time per day). The supernatant was collected and filtered through a 200 mesh sieve. Ethanol was removed when concentrated at 60° C., 600 mmHg. The sample was concentrated until 95% of volume was evaporated. The concentrate was then dried by freeze-drying at −57° C. under vacuum. The moisture content for the dried sample was 9-10%.

The powder so obtained was then used for the formulation of a preparation with the required concentration using conventional pharmaceutically accepted additives suitable for oral or parenternal administration to diabetic rats.

1.1.2 60% Ethanol Extracts

The bran was provided by Shanxi GAP cultivation site. Food Grade Ethanol was purchased from CSA Distilleries. Deionized water was provided from in house water system.

Buckwheat concentrates were produced by soaking the bran of tartary buckwheat in ten fold volume of 60% ethanol, reflux for 1 hour, 2 times repeated. The supernatant was collected and filtered through a 200 mesh sieve. Ethanol was removed when concentrated at 60° C., 600 mmHg. The sample was concentrated until 90% of volume was evaporated. The concentrate was then dried by spray-dried at inlet temperature of 140-150° C. and outlet temperature of 90-100° C. The moisture content for the dried sample was 3-5%, total flavonoids content was 25%, the yield was 10.4%.

1.1.3 Purified Flavonoids

A purified flavonoids A was purchased from Institute of Comprehensive Utilization of Agricultural Products, Shanxi Academy of Agricultural Science. The purity of this sample was 70%, and the moisture content was 3-5%.

A purified flavonoids B was prepared by our team. Tartary buckwheat bran concentrates were produced by soaking the bran of tartary buckwheat in ten fold volume of 60% ethanol, reflux for 1 hour, 2 times repeated. The supernatant was collected and filtered through a 200 mesh sieve. Ethanol was removed when concentrated at 60° C., 600 mmHg. The sample was concentrated until 90% of volume was evaporated. Let precipitate at 4° C. overnight, centrifuged at 3500 rpm for 20 minutes, collected the precipitate, then dried in vacuum oven at 60° C., 600 mmHg. The moisture content for the dried sample was 3-5%, total flavonoids content was 65%, yield 2.8%.

The powder so obtained is then used for the formulation of the required concentration using conventional pharmaceutically accepted additives suitable for oral or parenternal administration to diabetic rats.

1.1.4 Determination of Total Flavonoids Content

Flavonoids are plant polyphenols and were found in the buckwheat bran. Their hydrogen-donating antioxidant activity and their ability to complex divalent transition metal cations are known. Spectrometry method of measuring absorbance at 430 nm was employed for the determination of flavonoid content.

0.25-2.5 g sample was extracted by reflux in 125 ml 60% ethanol for 1 hour. The solution was filtered by vacuum. The residue was extracted again by reflux in 125 ml 60% ethanol for 1 hour and the solution was filtered. The filtrates were combined and the volume was fixed to 250 ml. The sample solution was freeze-dried and extract powder was obtained. The dried sample was dissolved in methanol to test.

1 ml of extract methanolic solution was used. 1 ml of methanolic AlCl3□ 6H2O was added. The sample was left for 10 minutes for reaction. Absorbance was measured at 430 nm.

0, 0.01, 0.02, 0.04, 0.06 mg/ml of Rutin standard was used. The test was conducted to obtain the absorbance (A). The standard solution concentration was used as x-axis while the absorbance (A) was used as y-axis. The regression formula was determined

The flavonoids content in samples was calculated through the regression formula.

1.2 Determination of Effect of the Extracts on the Alloxan Diabetic Mice

1.2.1 Animals

Male ICR mice, weighing 22 to 26 g, were purchased from VTLF Experiment Animal Technology Center Company Ltd., Beijing P. R. China. The animals were made diabetic by a single intravenous injection of 70 mg/kg alloxan into the tail vein. After a period of 4 days, the treated mice were fasted for 2 hours, then the blood sample was taken from the treated mice intravenously. The blood glucose concentration was determined using Glucose Oxidase Peroxidase method. The mice with the blood glucose concentration above 200 mg/dl were marked diabetic mice and were used for further experiments. A total of 27 mice were marked as diabetic mice.

1.2.2 Reagents

(1) 60% ethanolic extracts of tartary buckwheat bran as prepared in 1.1.2: 10.4% yield, 25% flavonoid

(2) 20% ethanolic extracts of tartary buckwheat bran as prepared in 1.1.1: 7.5% yield, 0.3% flavonoid

(3) Purified flavonoid B from tatary buckwheat bran as prepared in 1.1.3: 2.8% yield, 65% flavonoid

1.2.3 Methods

The mice were divided into 5 groups, each group having 9 mice. The difference in the average blood glucose concentration among the 5 groups was less than 10 mg/dl

Group 1 served as the negative control and received water, group 2 received metformin (200 mg/kg) as the positive control, groups 3 and 4 received reagents (1) and (2), respectively, at the dose of 5 g/kg (p.o.) (p.o. means oral administration), once every. Group 5 received reagent (3) at the dose of 5 g/kg (p.o.).

At day 7 and 14, measurements of blood glucose concentration were taken after fasting for 2.5 and 5 hours. The change of glucose concentration by percentage was compared against the control group. Blood triglyceride and cholesterol concentrations were tested at day 14 (enzymatic method). Glucose tolerance test (2.0 g/kg, p.o.) was conducted at day 19. First, the mice were fasted for half an hour before the administration of control and test substances. Two and half hours later, the mice received glucose at the dose of 2 g/kg, po. Blood samples were taken at 0, 30, 60, 120 minutes after administration of the glucose. Blood glucose concentration (mg/dl) of the mice were determined and the related measurements of Area Under the Curve (AUC) were estimated. The serum superoxide dismutase (SOD) level of blood samples taken at 0 minute was tested. Glucose tolerance test was conducted again at day 22. The mice were fasted for 1 hour before administration. One and half hour after administration, the mice received glucose at the dose of 2 g/kg, po Blood samples were taken at 0, 30 60, 120 minutes. For ease of description, the negative control group that received water is indicated simply as the “Control” while the positive control group is indicated as the “Metformin” group in the following tables.

1.2.4 Results

TABLE 1
The effects of tartary buckwheat bran extract on the blood glucose level
of alloxan diabetic mice

| | | Blood glucose concentration | | Blood glucose concentration | |
|---|---|---|---|---|---|
| | | at day 7 (mg/dl) | | at day 14 (mg/dl) | |
| | | 2.5 hr after | | 2.5 hr after | 5 hr after |
| | | administering | 5 hr after | administering | administering |
| | | (blood | administering | (blood | (blood |
| | | glucose | (blood glucose | glucose | glucose |
| | Dose | change by | change by | change by | change by |
| Group | (mg/kg) | percentage) | percentage) | percentage) | percentage) |
| Control | — | 357.9 ± 61.7 | 270.5 ± 63.7 | 357.1 ± 74.1 | 281.5 ± 24.6 |
| Metformin | 200 | 216.8 ± 78.2** | 182.8 ± 88.1* | 281.5 ± 60.9* | 221.7 ± 65.9 |
| | | (↓39.4) | (↓32.4) | (↓21.2) | (↓21.3) |
| 60% | 5000 | 323.8 ± 73.4 | 236.1 ± 74.0 | 394.2 ± 84.8 | 319.9 ± 91.6 |
| ethanol | | (↓9.5) | (↓12.7) | (↑10.4) | (↑13.6) |
| extract | | | | | |
| 20% | 5000 | 325.7 ± 72.9 | 227.7 ± 75.6 | 374.3 ± 63.0 | 325.4 ± 71.4 |
| ethanol | | (↓9.0) | (↓15.8) | (↑4.8) | (↑15.5) |
| extract | | | | | |
| Purified | 5000 | 335.6 ± 113.6 | 233.1 ± 83.2 | 387.1 ± 144.1 | 321.4 ± 125.5 |
| flavonoid B | | (↓6.2) | (↓13.8) | (↑8.4) | (↑14.1) |

Asterisks * indicate differences (*p < 0.05, **p < 0.01, ***p < 0.005) between control group and reagent-treated mice.
Numbers in bracket indicate the change of blood glucose concentration by percentage (%).

TABLE 2
The effects of tartary buckwheat bran extract on
the blood lipid level of alloxan diabetic mice at day 14

| | Dose | Blood cholesterol | Blood triglyceride |
|---|---|---|---|
| Group | (mg/kg) | (mg/dl) | (mg/dl) |
| Control | — | 173.4 ± 24.6 | 155.7 ± 33.9 |
| Metformin | 200 | 159.8 ± 48.7 | 132.8 ± 34.2 |
| 60% ethanol extract | 5000 | 190.0 ± 26.0 | 180.8 ± 29.5 |
| 20% ethanol extract | 5000 | 172.4 ± 16.5 | 133.9 ± 20.6 |
| Purified flavonoid B | 5000 | 172.4 ± 20.0 | 160.3 ± 27.2 |

TABLE 3
The effects of tartary buckwheat bran extract on the
serum SOD level of alloxan diabetic mice at day 19

| | | Serum SOD | |
|---|---|---|---|
| Group | Dose (mg/kg) | (u/mg) | SOD change (%) |
| Control | — | 70.0 ± 43.1 | 0 |
| Metformin | 200 | 78.3 ± 26.5 | ↑11.9 |
| 60% ethanol | 5000 | 115.0 ± 23.1* | ↑64.4 |
| extract | | | |
| 20% ethanol | 5000 | 94.8 ± 48.0 | ↑35.5 |
| extract | | | |
| Purified flavonoid B | 5000 | 85.5 ± 33.8 | ↑22.2 |

*indicates difference (p < 0.05) compared with the control group.

TABLE 4
The effects of tartary buckwheat bran extract on the glucose tolerance
and AUC of alloxan diabetic mice at day 19

| | Blood glucose (mg/dl) | | | | AUC |
|---|---|---|---|---|---|
| Group | 0 min | 30 min | 60 min | 120 min | (mg/dl · h) |
| Control | 396.5 ± 87.9 | 513.9 ± 88.2 | 457.6 ± 106.2 | 355.9 ± 137.3 | 877.2 ± 206.4 |
| Metformin | 346.8 ± 68.3 | 544.4 ± 45.3 | 512.0 ± 76.9 | 408.4 ± 71.3 | 947.1 ± 118.1 |
| 60% | 408.3 ± 75.2 | 554.9 ± 53.8 | 494.1 ± 65.8 | 440.7 ± 92.0 | 970.4 ± 131.6 |
| ethanol | | | | | |
| extract | | | | | |
| 20% | 362.0 ± 72.7 | 530.4 ± 58.7 | 462.4 ± 54.3 | 377.6 ± 83.8 | 891.3 ± 114.8 |
| ethanol | | | | | |
| extract | | | | | |
| Purified | 408.0 ± 129.8 | 517.2 ± 81.7 | 466.2 ± 75.4 | 394.7 ± 94.9 | 907.7 ± 167.5 |
| flavonoid B | | | | | |

TABLE 5
The effects of tartary buckwheat bran extract on the glucose tolerance
and AUC of alloxan diabetic mice at day 22

| | Blood glucose (mg/dl) | | | | AUC |
|---|---|---|---|---|---|
| Group | 0 min | 30 min | 60 min | 120 min | (mg/dl · h) |
| Control | 430.5 ± 66.1 | 520.1 ± 52.7 | 478.9 ± 53.7 | 387.2 ± 72.8 | 920.4 ± 113.4 |
| Metformin | 377.6 ± 87.6 | 474.5 ± 74.4 | 450.7 ± 80.1 | 358.6 ± 88.3 | 849.0 ± 158.3 |
| 60% | 381.5 ± 60.1 | 464.6 ± 38.7* | 438.9 ± 40.6 | 372.3 ± 56.2 | 843.0 ± 86.1 |
| ethanol | | | | | |
| extract | | | | | |
| 20% | 362.1 ± 54.4* | 428.1 ± 40.8*** | 391.3 ± 47.8** | 319.4 ± 73.6 | 757.7 ± 101.8** |
| ethanol | | | | | |
| extract | | | | | |
| Purified | 435.5 ± 103.2 | 444.2 ± 66.1* | 408.2 ± 54.6* | 339.4 ± 63.2 | 806.8 ± 125.0 |
| flavonoid B | | | | | |

*indicates differences (p < 0.05, **p < 0.01, ***p < 0.005) compared with the control group.

1.2.5 Conclusion

Neither the administration of a 20% ethanol tartary buckwheat extract nor of a 60% tartart buckwheat extract, continuously for 7 and 14 days at the dose of 5 g/kg, were effective in lowering blood glucose concentration in alloxan diabetic mice. However, metformin used in the positive control group showed significant glucose-lowering effect. After 7 and 14 days of continuing administration, the blood glucose concentration in alloxan diabetic mice treated by metformin was lowered by 34% (2.5 h, P<0.005), 32.4% (5 h, P<0.05), 21.2% (2.5 h, P<0.05), and 21.3% (5 h).

The administration of 20% and 60% ethanol extracts of tartary buckwheat, continuously for 14 days at the dose of 5 g/kg, did not affect the blood lipid level in alloxan diabetic mice.

Alloxan diabetic mice were treated separately with either a 20% ethanol extract of tartary buckwheat, a 60% ethanol extract of tartary buckwheat or purified flavonoids, continuously for 19 days at the dose of 5 g/kg. The treatments were effective in increasing the amounts of SOD in serum samples from the mice by 64.4% (P<0.05), 35.5%, and 22.2%, respectively.

These three treatments, however, were not effective in improving the glucose tolerance of alloxan diabetic mice when administered continuously for 19 days at the dose of 5 g/kg. This may be because glucose tolerance was tested 2.5 hours after administration and the effect of the doses of the treatments had begun diminishing. The glucose tolerance was tested again at day 22. The blood samples were taken 1.5 hour after administration. Results from blood taken at 0 min, 30 min and 60 min. showed that the 20% ethanol extract was effective at in lowering blood glucose and AUC at day 22. The 60% ethanol extract was also effective in lowering blood glucose as measured in blood samples taken at 30 and 60 minutes.

1.3 Determination of Effect of the Extracts on the Streptozocin (STZ) Diabetic Rats

1.3.1 Reagents

(1) Purified flavonoid A from tartary buckwheat as prepared in 1.1.3: 70% flavonoid

(2) Purified flavonoid B from tartary buckwheat as prepared in 1.1.3: 70% flavonoid

(3) 20% ethanolic extracts of tartary buckwheat bran as prepared in 1.1.1: 7.5% yield, 0.3% flavonoid

(4) 60% ethanolic extracts of tartary buckwheat bran as prepared in 1.1.2: 10.4% yield, 25% flavonoid

1.3.2 Animals

Male rats (Sprague-Dwaley, male), weighing 120 to 140 g, were purchased from VTLF Experiment Animal Technology Center Company Ltd., Beijing P. R. China The animals were made diabetic by a single intraperitoneal injection of streptozocin (freshly prepared in 0.1 M citrate buffer (pH 4.5) at the dose of of 58 mg/kg). After a period of 4 days, the treated rats were fasted for 2.5 hours, then blood samples were taken from the treated rats intravenously. Blood glucose concentrations were determined using the Glucose Oxidase Peroxidase method. The rats with blood glucose concentrations above 190 mg/dl were marked as diabetic rats and used for further experiments.

1.3.3 Methods

The rats were divided into 6 groups of 10 rats per group. The difference in the average blood glucose concentrations among the 6 groups was less than 10 mg/dl. Group 1 served as the negative control and received water. Group 2 received metformin (200 mg/kg) as the positive control. The remaining four groups received reagents (1) to (4) as listed in Section 1.3.1 above. Reagents (1) and (2) were administered at a dose of 4 g/kg, while (3) and (4) were administered at a dose of 5 g/kg. The treatments were administered once every day.

At day 7, the rats in groups 1, 2 and 3 were fasted and blood samples were taken at 2.5 hours and 5 hours after fasting began. Blood glucose concentrations (mg/dl) in these samples were then determined.

At day 14, glucose tolerance tests were conducted. The rats were fasted for 1 hour before receiving the reagents at dosages of 2.0 g/kg (p.o.). Blood samples were taken at 0, 30, 60, and 120 min after receiving the reagents. Blood glucose concentrations (mg/dl) were determined and the related AUC were calculated.

At day 19, blood samples were taken, and blood glucose concentrations without fasting, the activities of SOD and catalase (CAT) levels were determined.

At day 21, glucose tolerance tests were conducted. The rats were fasted for 1 hour, then received their respective reagents at the dose of 5 g/kg (p.o.). 2.5 hours after administration of reagent the rats received glucose at a dose of 2 g/kg (p.o.). Blood samples were taken at 0, 30, and 120 minutes after glucose administration. Blood glucose concentrations (mg/dl) were determined and the related AUC were calculated.

1.3.4 Results

TABLE 6
The effects of tartary buckwheat bran extract on the
fasting blood glucose concentration in STZ diabetic
rat after oral administration for 7 days

| | | Fasting blood glucose | |
|---|---|---|---|
| | | concentration (mg/dl) | |
| Sample | dose (g/kg) | 2.5 hr. | 5.0 hr. |
| Control | — | 312.3 ± 53.5 | 282.6 ± 34.4 |
| Metformin | 0.2 | 261.1 ± 45.7* | 185.6 ± 78.8** |
| | | (↓16.4) | (↓34.3) |
| Purified | 4.0 | 2.4.9 ± 37.7 | 244.6 ± 77.8 |
| flavonoid A | | (↓5.6) | (↓13.5) |
| Purified | 4.0 | 311.2 ± 18.6 | 272.6 ± 26.0 |
| flavonoid B | | (↓0.4) | (↓3.5) |
| 20% ethanol | 5.0 | 277.0 ± 35.3 | 247.5 ± 27.4* |
| extract | | (↓11.3) | (↓12.4) |
| 60% ethanol | 5.0 | 311.8 ± 41.2 | 258.6 ± 15.7 |
| extract | | (↓0.2) | (↓8.5) |

Asterisks * indicate differences (*p < 0.05, **p < 0.01) compared with the control group. Numbers in the brackets indicate the change of blood glucose concentration by percentage (%).

At day 7, the fasting blood glucose concentration in STZ diabetic rats that received reagent (3) at a dose of 5 g/kg was significantly reduced by 12.4%. Those rats that received reagents (1) and (2) (at 4 g/kg) and (4) (at 5 g/kg) did not show any significant effects.

TABLE 7
The effects of tartary buckwheat bran extracts on blood glucose concentration and
AUC in STZ-diabetic rat after oral administration for 14 days

| | dosage | blood glucose concentration (mg/dl) | | | | AUC |
|---|---|---|---|---|---|---|
| Sample | (g/kg) | 0 min | 30 min | 60 | 120 min | (mg/dl · hr) |
| Control | — | 398.4 ± 75.0 | 473.0 ± 104.5 | 438.9 ± 75.5 | 355.1 ± 62.0 | 842.8 ± 150.1 |
| Metformin | 0.2 | 341.8 ± 44.8 | 356.2 ± 93.3* | 320.9 ± 64.6* | 296.6 ± 72.6 | 652.5 ± 133.8* |
| | | | | | | (↓22.6) |
| Purified | 4.0 | 370.1 ± 55.5 | 421.5 ± 68.0 | 378.9 ± 83.9 | 339.9 ± 82.6 | 757.3 ± 139.5 |
| flavonoid A | | | | | | (↓10.1) |
| Purified | 4.0 | 364.1 ± 25.2 | 440.5 ± 39.4 | 406.6 ± 31.0 | 367.9 ± 26.7 | 800.2 ± 53.8 |
| flavonoid B | | | | | | (↓5.1) |
| 20% | 5.0 | 354.5 ± 31.7 | 382.3 ± 40.4* | 359.0 ± 51.5* | 337.1 ± 37.8 | 717.6 ± 75.8* |
| ethanol | | | | | | (↓14.9) |
| extract | | | | | | |
| 60% | 5.0 | 364.7 ± 37.0 | 400.4 ± 36.1 | 378.9 ± 42.1 | 328.7 ± 36.0 | 739.6 ± 69.5 |
| ethanol | | | | | | (↓12.2) |
| extract | | | | | | |

Asterisks * indicate differences (*p < 0.05) compared with control group.
Numbers in brackets indicate the reduction of AUC by percentage (%)

Table 7 shows the effects of tartary buckwheat bran extracts (as prepared according to the method described in section 1.1). At day 14, glucose tolerance test was conducted. Results showed that the 20% ethanol extract was effective in lowering the blood glucose concentration at 30 min and 60 min and its AUC was lower than the control group by 14.9%.

TABLE 8
The effects of tartary buckwheat bran extract on blood
glucose concentrations without fasting, serum SOD
activity and catalase (CAT) activity in STZ-diabetic rat
after oral administration for 19 days

| | | Non-fasting blood | | |
|---|---|---|---|---|
| | Dosage | glucose | Serum SOD activity | CAT activity |
| Group | (g/kg) | concentration mg/dl) | (NU/ml) | (U/gHb) |
| Control | — | 361.5 ± 58.2 | 121.6 ± 32.8 | 68.4 ± 22.4 |
| Metforimin | 0.2 | 340.5 ± 40.7 | 164.8 ± 16.9** | 64.6 ± 12.5 |
| | | (↓5.8) | (↑35.3) | (↓5.5) |
| Purified | 4.0 | 324.0 ± 91.4 | 112.2 ± 24.4 | 79.8 ± 15.3 |
| flavonoid A | | (↓10.4) | (↓7.8) | (↑16.7) |
| Purified | 4.0 | 304.3 ± 69.7 | 86.9 ± 8.0** | 73.2 ± 23.6 |
| flavonoid B | | (↓15.8) | (↓28.6) | (↑7.0) |
| 20% ethanol extract | 5.0 | 264.1 ± 55.6** | 132.5 ± 17.7 | 98.3 ± 16.3** |
| | | (↓26.9) | (↑9.0) | (↑43.7) |
| 60% ethanol extract | 5.0 | 298.4 ± 36.5* | 158.7 ± 18.2** | 86.0 ± 17.1 |
| | | (↓17.4) | (↑30.5) | (↑25.7) |

Asterisks * indicate differences (*p < 0.05, **p < 0.01) compared the control group. Number in the brackets indicate the change by percentage.

At day 19, rats treated with reagent (3) (at 5 g/kg) showed a significant effect by lowering blood glucose concentrations by 26.9% and increasing the activity of CAT by 43.7% in comparison to the control group. Rats treated with reagent (4) (at 5 g/kg) also showed a significant effect by lowering blood glucose concentrations by 17.4% and increasing the activity of serum SOD by 30.5%. Reagents (1) and (2) were not effective on non-fasting blood glucose concentrations and CAT activities and reagent (2) (at 4 g/kg) actually reduced the activity of serum SOD by 28.6%, in comparison to the control group.

TABLE 9
The effects of tartary buckwheat bran extracts on oral glucose tolerance in STZ
diabetic rat after oral administration for 21 days

| | dosage | blood glucose concentration(mg/dl) | | | AUC |
|---|---|---|---|---|---|
| Sample | (g/kg) | 0 min | 30 min | 120 min | (mg/dl · hr) |
| Control | — | 363.4 ± 61.1 | 463.9 ± 56.6 | 338.3 ± 55.8 | 808.5 ± 101.9 |
| Metformin | 0.2 | 309.8 ± 68.5 | 358.0 ± 77.3** | 279.8 ± 51.6* | 645.4 ± 122.0** |
| | | | | | (↓20.2) |
| 20% | 5.0 | 322.8 ± 59.4 | 379.6 ± 64.9** | 327.4 ± 32.6 | 705.9 ± 95.2* |
| ethanol | | | | | (↓12.7) |
| extracts | | | | | |

Asterisks * indicate differences (*p < 0.05, **P < 0.01) compared with the control group.
Numbers in brackets indicate the change of AUC by percentage (%).

At day 21, glucose tolerance tests were conducted. The 20% ethanol extracts lowered blood glucose concentrations by 64.9% and reduced AUC values by 12.7% at 30 min, in comparison to the control group.

TABLE 10
The effects of tartary buckwheat bran extracts on weight in
STZ diabetic rat after oral administration for 9, 14 and 21 days

| | dosage | weight(g) | | |
|---|---|---|---|---|
| Sample | (g/kg) | At day 9 | At day 14 | At day 21 |
| Control | — | 216.2 ± 20.3 | 217.3 ± 25.4 | 191.5 ± 31.6 |
| Metformin | 0.2 | 203.3 ± 22.3 | 200.4 ± 29.4 | 185.3 ± 27.3 |
| Purified | 4.0 | 203.6 ± 36.3 | 199.7 ± 42.8 | N/A |
| flavonoid A | | | | |
| Purified | 4.0 | 198.5 ± 36.7 | 210.5 ± 33.7 | N/A |
| flavonoid B | | | | |
| 20% ethanol | 5.0 | 224.3 ± 13.1 | 237.5 ± 18.5 | 225.1 ± 33.8* |
| extracts | | | | |
| 60% ethanol | 5.0 | 220.1 ± 24.1 | 234.0 ± 32.7 | N/A |
| extracts | | | | |

Asterisks * indicate differences (*p < 0.05) compared with the control group.

Before administration, there was no obvious difference among the rats of different groups. After administration for 21 days, the average weight of rats receiving 20% ethanol extract was significantly heavier than the control group.

1.3.5 Conclusion

When administered at the dose of 5 g/kg continuously for 7 days, the 20% ethanol extract of tartary buckwheat was effective in lowering the fasting blood glucose concentration 5 hours after administration. After 14 and 21 days of continuous administration, the glucose tolerance of STZ diabetic rats was also significantly improved. After 19 days of continuous administration, the non-fasting blood glucose concentrations of STZ diabetic rats was lowered and the serum CAT activities were enhanced. The average weight of the rats receiving 20% ethanol extract at 5 g/kg was heavier than the control group after treatment. Their overall condition was also better than the control group. The above results demonstrate that 20% ethanol extract of tartary buckwheat was effective for the amelioration of diabetic symptoms of STZ diabetic rats.

At the dose of 5 g/kg, the 60% ethanol extract of tartary buckwheat was effective in lowering the non-fasting blood glucose of STZ diabetic rats after 19 days of continuous administration. It was also effective in enhancing the serum SOD and the CAT activities. However, it was not effective in improving fasting blood glucose levels or glucose tolerance.

Flavonoid A (4 g/kg) and B (4 g/kg) purified from tartary buckwheat do not affect fasting blood glucose and non-fasting blood glucose concentrations, CAT activities or glucose tolerance. Flavonoid B (4 g/kg) produced a negative effect by reducing serum SOD activity.

Flavonoid A and B were administered at 4 g/kg instead of 5 g/kg as used in other groups. This was due to flavonoid A becoming too condensed for administration when used at a concentration of 5 g/kg.

In conclusion, the 20% ethanol extracts of tartary buckwheat were the most effective reagents among those tested for the amelioration of hyperglycemic and diabetic symptoms.

### EXAMPLE 2

### Further Analysis of 20% Ethanol Extract of Tartary Buckwheat on STZ Diabetic Rats

In this sets of experiments, a range of doses of the 20% ethanol extract of tartary buckwheat administered for a variety of lengths of time were tested for effectiveness in the treatment of STZ diabetic rats.

2.1 Reagent

20% ethanol extract of tartary buckwheat as prepared in 1.1.1: 7.5% yield; 0.3% flavonoid.

2.2 Animals

The mice were divided according to the blood glucose concentration under fasting conditions. The difference between the average blood glucose concentrations of the 4 groups of rats was less than 10 mg/dl.

2.3 Methods

The STZ diabetic rats were divided into 4 groups, each group having 10 rats. Group 1 served as the negative control and received water, group 2 received metformin (200 mg/kg) as the positive control, group 3 received 20% ethanol extract of the bran of tartary buckwheat seeds at the dose of 2.5 g/kg (p.o.), group 4 received 20% ethanol extract of the bran of tartary buckwheat seeds at the dosage of 5 g/kg (p.o.), once every day for 12 days in a row.

At day 7, fasting blood glucose concentration was tested after 2.5 and 5 hours of fasting. The change of glucose concentration by percentage was compared against the control group.

At day 12, oral glucose tolerance tests (2.0 g/kg) were conducted. The rats were fasted for 1 hour before administration of reagents at dosages of 2.5 or 5 g/kg (p.o.). 1 hour later, the rats received glucose at the dose of 2 g/kg (p.o.). Blood samples were taken at 0, 30 and 120 min respectively. Blood glucose concentrations (mg/dl) were determined and the related AUC values were calculated.

2.4 Results

TABLE 11
The effects of 20% ethanol extract of tartary buckwheat
on the fasting blood glucose concentration in STZ diabetic
rats after oral administration for 7 days

| | |
|---|---|
| | fasting blood glucose |
| dosage | concentration (mg/dl) |

| Group | (g/kg) | 2.5 hr. | 5 hr. |
|---|---|---|---|
| Control | — | 404.6 ± 40.2 | 372.7 ± 59.3 |
| metformin | 0.2 | 357.2 ± 51.5 | 288.9 ± 68.8* |
| | | (11.7) | (22.5) |
| 20% Extract | 2.5 | 340.8 ± 44.5** | 310.6 ± 37.3* |
| | | (15.8) | (16.7) |
| 20% Extract | 5.0 | 329.0 ± 43.0** | 311.3 ± 21.5* |
| | | (18.7) | (16.5) |

Asterisks * indicate differences (*p < 0.05, **p < 0.01) compared with the control group, numbers in brackets indicate the reduction of blood glucose concentration by percentage (%).

After oral administration for 7 days, the rats in group 1, 2, 3 and 4 were fasted for 2.5 and 5 hours, blood samples were taken, and blood glucose concentrations (mg/dl) of these rats were determined. The 20% extract was effective in lowering blood glucose concentration by 15.8%, 18.7%, 16.7%, and 16.5% (Table 11).

TABLE 12
The effects of tartary buckwheat bran extract on oral glucose
tolerance in STZ diabetic rats after 12 days oral administration

| | | Fasting blood glucose | | | |
|---|---|---|---|---|---|
| | dosage | concentration (mg/dl) | | | AUC |
| Group | (g/kg) | 0 min | 30 min | 120 min | (mg/dl · hr) |
| Control | — | 448.6 ± 52.2 | 475.2 ± 52.6 | 408.3 ± 41.9 | 934.0 ± 80.8 |
| metformin | 0.2 | 398.4 ± 36.6 | 458.4 ± 59.7 | 323.9 ± 51.2* | 800.9 ± 103.2* |
| | | | | | (14.2) |
| 20% extract | 2.5 | 399.0 ± 56.4 | 455.6 ± 69.6 | 359.5 ± 63.8 | 825.0 ± 124.8 |
| | | | | | (11.7) |
| 20% extract | 5.0 | 434.2 ± 27.6 | 456.3 ± 32.4* | 357.9 ± 53.9 | 833.3 ± 17.5** |
| | | | | | (10.8) |

Asterisks * indicate differences (*p < 0.05, **P < 0.01) compared with the control group. Numbers in brackets indicate the reduction of AUC by percentage (%).

After oral administration for 12 days, glucose tolerance tests on rats given extracts or control substances were conducted. The 20% ethanol extract at the dose of 5 g/kg showed a statistically significant (p<0.05) anti-diabetic effect at 30 min by lowering the blood glucose concentrations and AUC (10.8%) values in comparison to the control group (water) of rat (Table 12).

2.5 Conclusion

The 20% ethanol extract of tartary buckwheat was effective in lowering the fasting blood glucose of STZ diabetic rats 2.5 and 5 hours after single dose administration when administered over the previous consecutive 7 days at the doses of 2.5 g/kg and 5 g/kg. After administration for 12 days at the doses of 2.5 g/kg and 5 g/kg, the glucose tolerances of the STZ diabetic rats were also improved with different degrees. The effects of differing amounts of the extract on blood glucose concentrations were basically the same.

### CONCLUSION

Based on the pharmacology results, the results above show that 20% ethanol extract of tartary buckwheat has effect on hyperglycemia or diabetes in animal models. The data indicate that in a preferred embodiment, a dose equivalent to 18.2 g human daily dosage is effective for treatment of hyperglycemia or diabetes in humans.

While the present invention has been described with respect to the effectiveness of tartary buckwheat extracts on lowering glucose concentrations in mice/rats, it should be understood by those skilled in the art that various changes may be made and equivalents may be substituted without departing from the true spirit and scope of the invention. For instance, the ethanol used can be replaced with other alcohols, such as propanol, isopropanol, n-butanol, etc. In addition, many modifications may be made to adapt a particular situation, material, composition of matter, process, process step or steps, to the objective, spirit and scope of the present invention. All such modifications are intended to be within the scope of the claims appended hereto. As used in the present specification and claims, the terms “comprise,” “comprises,” and “comprising” mean “including, but not necessarily limited to”. For example, a method, apparatus, molecule or other item which contains A, B, and C may be accurately said to comprise A and B. Likewise, a method, apparatus, molecule or other item which “comprises A and B” may include any number of additional steps, components, atoms or other items as well.

### REFERENCE

The following references are incorporated herein by reference in their entireties:

Kayashita, J., Shimaoka, I., Nakajoh, M., Kishida, N., & Kato, N. (1999). Comsumption of a buckwheat protein extract retards 7,12-dimethylbenz[alpha]anthracene-induced mammary carcino-genesis in rats. Bioscience, Biotechnology, and Biochemistry, 63, 1837-1839.

Kreft, I., Bonafaccia, G., & Zigo, A. (1994). Secondary metabolites of buckwheat and their importance in human nutrition. Prehrambenotehnoloska i Biotechnoloska Revija, 32, 195-197.

Kreft, I. Skrabanja, V., Ikeda, S. Ikeda, K., & Bonafaccia, G. (1996). Dietary value of buckwheat. Zbornik BFUL, 67, 73-78.

Liu, Z., Ishikawa, W., Huang, X., Tomotake, H., Kayashita, J., Watanabe, H., Nakajoh, M., & Kato, N. (2001). A buckwheat protein product suppresses 1,2-dimethylhydrazine-induced colon carcinogenesis in rats by reducing cell proliferation. The Journal of Nutrition, 131, 1850-1853.

Skrabanja, V., Laerke, H. N., & Kreft, I. (1998). Effects of hydrothermal processing of buckwheat (Fagopyrum esculentum Moench) groats on starch enzymatic availability in vitro and in vivo in rats. Journal of Cereal Science, 28, 209-214.

Skrabanja, V. Liljeberg, E. H. G. M., Kreft, I., & Bjorck, I. M. E. (2001). Nutritional properties of starch in buckwheat products: studies in vitro and in vivo. Journal of Agricultural and Food Chemistry, 49, 490-496.

Steadman, K. J., Burgoon, M. S., Lewis, B. A., Edwardson, E. E., & Obendorf, R. L. (2001). Buckwheat seed milling fractions: description, macronutrient composition and dietary fibre. Journal of Cereal Science, 33, 271-278.

What is claimed is:

1. A product obtained by extracting buckwheat with about 10-30% ethanol.

2. The product of claim 1, wherein about 20% ethanol is used for said extracting.

3. The product of claim 1, wherein said buckwheat is tartary buckwheat.

4. The product of claim 1, wherein said buckwheat is buckwheat bran.

5. The product of claim 1, wherein said product is obtained by centrifugation of said about 10-30% ethanol after said about 10-30% ethanol has been placed in contact with said buckwheat.

6. The product of claim 1, wherein said extraction further comprises concentration of said product by a process selected from the group consisting of evaporation and drying.

7. A pharmaceutical composition for lowering blood glucose comprising a extract of buckwheat bran obtained by extraction with about 10% to 30% ethanol.

8. A nutritional supplement for lowering blood glucose comprising an extract of buckwheat bran obtained by extraction with about 10% to 30% ethanol.

9. A method for treating diabetic symptoms in an individual comprising the administration of an effective dose of an extract of buckwheat to said individual, wherein said extract was obtained by extraction of buckwheat with about 10% to 30% ethanol.

10. The method of claim 9, wherein said ethanol is about 20% ethanol.

11. The method of claim 9, wherein said diabetic symptoms are selected from the group consisting of hyperglycemia, glucose intolerance and hyperlipidemia.

12. The method of claim 11, wherein said hyperlipidemia is selected from the group consisting of hypercholesterolemia and hypertriglyceridemia.

13. The method of claim 9, wherein said treatment manifests as an effect selected from the group consisting of lower fasting blood glucose, lower non-fasting blood glucose, higher levels of superoxide dismutase activity, higher catalase activity levels, lowering peak glucose levels and increasing the rate of falling blood glucose levels.

14. A method for isolating an anti-hyperglycemic agent from buckwheat, comprising:
(a) drying seed from said buckwheat;
(b) isolating bran from said seed;
(c) extracting said agent from said bran with about 10% to 30% ethanol; and
(d) processing said ethanol to isolate said anti-hyperglycemic agent.

15. The method of claim 14, wherein said buckwheat is tartary buckwheat.

16. The method of claim 14, wherein said drying of said seed takes place at a temperature of about 10 degrees Celsius.

17. The method of claim 14, wherein said alcohol is about 10% to 30% ethanol (w/v).

18. The method of claim 17, wherein said ethanol has a concentration of about 20% (w/v).

19. The method of claim 14, wherein said processing comprises:
(a) centrifuging said ethanol to obtain a supernatant liquid;
(b) evaporating said supernatant liquid to obtain a residue; and
(c) drying said residue.

20. The method of claim 19, wherein said centrifuging is performed at speeds of about 1000 to about 3000 RPM for about 5 to about 30 minutes.

21. The method of claim 19, wherein said method further comprises a filtering step after centrifugation.

22. The method of claim 21, wherein said filtering step is performed with an about 100 to about 300 mesh sieve filter.
